SEQUENCE LISTING (1 sequence)

SEQ ID NO: 1 - DNA, 1194 bp, Pseudomonas aeruginosa
atgtggtatcacggcatcttacagccaacgctgtggcaactgattgtcgcgactttggcgctgacccatgtcagtatcatcagtgtgacgctatatctgcatcgccattcagctcatcgctcggtgcaattgcaccctgcgctaaagcacttcttccgtttctggctgtggctgacgacgggaatgaacacccgtgcgtggacggccgttcatcgccggcatcatgcctgctgcgaaacgcccgacgatccgcacagcccgaggttcaaggggctgctcacggtgttgctgcgaggtgccgagctgtatagagccgaggctcgcaatccggaaaccttgcagacctatggccgaggctgcccggacgactggctggagcgtcgcctctacagtaggtttccgaacgccgggctgggcctcatgctcggtgtggacttggttttattcggcgtcgcggggctgacagtctgggcgctgcagatgatctggatcgccttctgggctgccggtgtgatcaacggtctgggccatgctttcggctatcgcaccttcgaatgcccggacgattcgagcaatctgctgccgtggggagtcctcgtcggcggcgaggagttgcataacaaccatcatgcctacccgggctcggccaagctttccgtgcaggcctgggagtttgatctgggctgggcgtggatatgtctgctgagagcactcggactggctcaggtcaaccgtgggctgcccaccatccagtgggactgtgacaagcgagcgctggatctggaagccagcggagtcatcctgcgcaatcgtctgctggtcatggcccaatacgggaaacgggtaatgcggccgctggcacgcgaagaaagcgctcgctgtgatgcggcgacccggcatttgctgcggcgcgccagccgtttgctggtgcgggacccagacaggctgagcgatctgcagcagagcctgttccattcagctgtcggcaggagccaggtgatgcagctagcctgcgcgcatcgcctggcgttacagcgcacatggagcgatgcgggggctgacctgggcgaagtacatgcggcgttgaaacgctgggtaaccgaggcggaagccagcgggatacgtccgttgagggagtttgccgggctactgtgcagctacagcctgtacgctccgcggcagatcgtgccgtag

(19) United States
(12) Patent Application Publication
(10) Pub. No.: US 2016/0347989 A1
(43) Pub. Date: Dec. 1, 2016

(54) MIXED BACTERIA PRODUCING BIOSURFACTANT AND ITS SCREENING METHOD

(71) Applicant: ZFA Technologies Inc., Suzhou City (CN)

(72) Inventor: Hao DUAN, Suzhou City (CN)

(73) Assignee: ZFA Technologies Inc., Suzhou City (CN)

(21) Appl. No.: 15/141,349

(22) Filed: Apr. 28, 2016

(30) Foreign Application Priority Data
May 29, 2015 (CN) 201510170685.9

(51) Int. Cl. C09K 8/584; C12Q 1/02; C12N 1/20

(52) U.S. Cl. CPC C09K 8/584; C12N 1/20; C12Q 1/02; G01N 2333/21; G01N 2333/195; G01N 2333/32; G01N 2405/00

(57) ABSTRACT

Embodiments of the invention relate to a mixed bacteria producing surfactant and its screening method. According to at least one embodiment, there is provided a mixed bacteria producing biosurfactant composed of three kinds of strains: Pseudomonas stutzeri with preservation No. CCTCC AB 205091, Nocardioides ginsengagri with preservation No. CCTCC S2013441, and Bacillus licheniformis with preservation No. CCTCC AB 205141. The mixed bacteria is obtained by choosing the bacterial strain for oil production awaiting screening, activating and culturing it, getting the fermentation liquid in primary screen with blood plate method, and re-screening fermentation liquid in primary screen with oil drain method. This mixed bacteria synthesizes the advantages of the three strain, thus producing a biosurfactant to enhance oil recovery in the oilfield. The screening method of this mixed bacteria has a broad scope of applications, which effectively reduce the screening cost with high accuracy, provides strong selectivity, is a convenient process and easy to operate, and has a shorter cycle compared with the current technology.

### CROSS REFERENCE TO RELATED APPLICATION

This application claims the benefit of and priority to Chinese Patent Application Serial No. 201510170685.9, filed on May 29, 2015, entitled (translation), “MIXED BACTERIA PRODUCING BIOSURFACTANT AND ITS SCREENING METHOD,” which is hereby incorporated by reference in its entirety into this application.

### BACKGROUND

Field of the Invention

Embodiments of the invention relate to a microorganism used in the oilfield for oil recovery, especially a mixed bacteria producing biosurfactant and its screening method.

Description of the Related Art

Enhanced oil recovery is a recovery technology used to improve oil recovery by improving the physical and chemical properties of reservoir and reservoir fluid. The oil exploitation technology consists of primary oil recovery, secondary oil recovery, and tertiary oil recovery. Primary oil recovery is conducted by using the natural energy in the reservoir; secondary oil recovery develops an oilfield by recovering the reservoir pressure with certain technology, and among which waterflooding for oil recovery is most commonly used. Thereafter, the technology of enhancing oil recovery belongs to tertiary oil recovery. At present, major oilfields in oil producing countries in the world have come to a later development period apart from Middle East. Therefore, it is of great significance to develop all kinds of new tertiary oil recovery technology, to enhance well recovery for improving crude oil production, and to ease the oil crisis.

Microorganisms plays an important role in oil exploration, oil recovery, and oil environmental protection. China has tackled the microbial enhanced oil recovery in the Ninth and Tenth Five Year Plan. Additionally, Sinopec Group and Chinese Academy of Sciences cooperate conduct extensive indoor basic research and field application research on oil microorganism technology in Shengli Oilfield, where an oil microbial strain database and matered strain of more than 120 various uses have been established. Oil microorganism technology has strong adaptability and wide application prospects during the production process in the oil industry, which could not only enhance oil recovery and prolong oilfield development lifetime, but also degrade oil pollutants, solving the crude oil pollution in the production process of oilfields without secondary pollution. At present, the oil recovery is about 50% in the oil production industry in the world. The research of the United States Department of Energy shows that microorganisms could enhance oil recovery 10%-15%, while prolonging reservoir development period 5-10 years. The Statoil ASA in Norway has used MEOR in the Nome Oilfield with the result of increased production of 7%-10% and an expectation of cumulative increased crude oil production of 30 Million bbls in 15 years. In addition, the MEOR experiment in Shengli Oilfield has an accumulative increased oil production of more than 60,000 bbls.

Up to now, major oil producing countries in the world have regarded the MEOR as the main research project in the new generation. New biosurfactant preparations could apply to the technologies of perforation, operation wash well, well completion, etc. to prevent plant residue damage, clean grease viscosity, and induced oil flow while harmless to the reservoir. Oil well production has increased above 30% than any comparable wells on the basis of better protecting the environment with this technology in well completion. However, no obvious breakthrough has been made in the application of biosurfactant produced by microorganisms to the oilfield construction technology.

### SUMMARY

Embodiments of the invention provide a screening method for a mixed bacteria producing biosurfactant of highly efficient biological surface active catalytic agents formed by biochemical extraction.

According to at least one embodiment, there is provided a mixed bacteria producing biosurfactant, including three strains being comprised of (1) Pseudomonas stutzeri, preservation No.: CCTCC AB 205091, (2) Nocardioides ginsengagri, preservation No.: CCTCC S2013441, and (3) Bacillus licheniformis, preservation No.: CCTCC AB 205141.

According to another embodiment, there is provided a screening method of a mixed bacteria producing biosurfactant, including the steps of: selecting a bacterial strain for oil production awaiting screening; activating and culturing the selected bacterial strain for oil production awaiting screening; selecting the bacterial strain for oil production awaiting screening which is activated and cultured to obtain a primary screening fermentation liquid using a blood plate method; and re-screening the primary screening fermentation liquid to obtain the mixed bacteria producing biosurfactant using an oil drain method.

According to at least one embodiment, the selected bacterial strain is comprised of three strains including Pseudomonas stutzeri with preservation No. of CCTCC AB 205091, Nocardioides ginsengagri with preservation No. of CCTCC S2013441, and Bacillus licheniformis with preservation No. of CCTCC AB 205141.

According to at least one embodiment, the activating step includes transferring the bacterial strain for oil production awaiting screening onto a slant medium, and culturing the slant medium for 2 days at 37° C., wherein the slant medium is comprised of beef extract 3 g, peptone 10 g, NaCl 5 g, agar 20 g, and distilled water 1000 mL, and has a pH of 7.0, and a steam sterilization of 121° C., 20 minutes.

According to at least one embodiment, the culturing step includes seed culturing and fermentation culturing in order.

According to at least one embodiment, the seed culturing step includes transferring the bacterial strain's seed awaiting screening, after the activation and culturing the selected bacterial strain, to a seed liquid medium, shaking the cultivation at 37° C. for 16 hours using a rotational speed of 160 revolutions/minute, wherein the seed liquid medium is comprised of grape 5 g, beef extract 3 g, peptone 10 g, MgSO₄.7H₂O 2 g, and deionized water 1000 mL, and has a pH of 7.2, and a steam sterilization of 121° C., 20 minutes.

According to at least one embodiment, the fermentation culturing step includes inoculating a 4% inoculation amount of a seed liquid produced by seed culturing in a primary fermentation medium, shaking the cultivation at 37° C. for 72 hours using a rotational speed of 160 revolutions/minute, wherein the primary fermentation medium is comprised of glucose 20 g, peptone 4 g, KH₂PO₄ 5 g, K₂HPO₄ 5 g, MgSO₄.7H₂O 0.25 g, NaCl 2 g, CaCl₂ 0.08 g, and deionized water 1000 mL, and has a pH of 7.0, and a steam sterilization of 121° C., 20 minutes.

According to at least one embodiment, the blood plate method includes dibbling the bacterial strain awaiting screening with a sterile toothpick on a cooling blood plate separation culture medium, culturing the bacterial strain for 24 h-48 h at 37° C. to determine the bacterial strain's ability to produce biosurfactant according to a hemolysis ring diameter on the blood plate, wherein the blood plate separation culture medium is comprised of beef extract 3 g, peptone 10 g, NaCl 5 g, agar 15 g-20 g, distilled water 1000mL, and has a pH of 7.0-7.2, and a steam sterilization of 121° C., 20 minutes; the blood plate method further comprising, adding 100 mL of supernatant of fresh pig blood and homogeneously mixing, when the blood plate separation culture medium is cooled to about 45° C.

According to at least one embodiment, the oil drain method includes fetch dibbling the bacterial strain awaiting screening and a blood plate separation culture vessel with diameter 15 cm, adding 1 mL of a liquid paraffin after adding 100 mL water, when the liquid paraffin spreads into a circular oil film, adding 10 μL a fermentation liquid that has centrifugal, removed impurities, after extraction in the center, and measuring the diameter of an oil drain ring and tracking measurements for 5 days.

According to another embodiment, there is provided a biosurfactant extracted and processed by mixed bacteria according to the screening method generally discussed above and described in more detail below.

According to at least one embodiment, the main ingredient of the biosurfactant is rhamnolipid.

According to at least one embodiment, the physical and chemical index of the biosurfactant is a concentrated liquid having a brown translucent appearance and enzyme odor, a PH of 5-7, a density of 1-1.1 g/cm³, is completely soluble in water and compatible with any degree of mineralization of sewage, is insoluble in oil, and has a boiling point 100° C. and temperature resistance ≦220° C.

According to another embodiment, there is provided an application of the biosurfactant extracted and processed by mixed bacteria generally discussed above and described in more detail below in an oilfield for recovery of oil.

According to at least one embodiment, the application density of the biosurfactant is 0.5-3.0wt. %.

According to at least one embodiment, the application density of the biosurfactant is 2.0 wt %.

According to at least one embodiment, the biosurfactant is applied to a working environment no higher than 220° C.

According to at least one embodiment, the biosurfactant is applied to enhance production for a heave oil thermal recovery well.

### BRIEF DESCRIPTION OF THE DRAWINGS

So that the manner in which the features and advantages of the invention, as well as others which will become apparent, may be understood in more detail, a more particular description of the invention briefly summarized above may be had by reference to the embodiments thereof which are illustrated in the appended drawings, which form a part of this specification. It is to be noted, however, that the drawings illustrate only various embodiments of the invention and are therefore not to be considered limiting of the invention's scope as it may include other effective embodiments as well.

FIG. 1 shows the growth curve of bacteria strain DN4-3 according to an embodiment of the invention.

FIG. 2 shows the seed age's effect to fermentation of lipopeptide according to an embodiment of the invention.

FIG. 3 shows the static oil wash effect without adding biosurfactant according to an embodiment of the invention.

FIG. 4 shows the static oil wash effect with 2% biosurfactant according to an embodiment of the invention.

FIG. 5 shows the oil sand after static oil wash without biosurfactant according to an embodiment of the invention.

FIG. 6 shows the oil sand after static oil wash with 2% biosurfactant according to an embodiment of the invention.

### DETAILED DESCRIPTION

Although the following detailed description contains many specific details for purposes of illustration, it is understood that one of ordinary skill in the relevant art will appreciate that many examples, variations, and alterations to the following details are within the scope and spirit of the invention. Accordingly, the exemplary embodiments of the invention described herein are set forth without any loss of generality, and without imposing limitations, relating to the claimed invention. Like numbers refer to like elements throughout. Prime notation, if used, indicates similar elements in alternative embodiments.

Embodiments of the invention relate to a mixed bacteria producing surfactant and its screening method. According to at least one embodiment, there is provided a mixed bacteria producing biosurfactant composed of three kinds of strains: Pseudomonas stutzeri with preservation No. CCTCC AB 205091, Nocardioides ginsengagri with preservation No. CCTCC S2013441, and Bacillus licheniformis with preservation No. CCTCC AB 205141. The mixed bacteria is obtained by choosing the bacterial strain for oil production awaiting screening, activating and culturing it, getting the fermentation liquid in primary screen with blood plate method, and re-screening fermentation liquid in primary screen with oil drain method. This mixed bacteria synthesizes the advantages of the three strain, thus producing a biosurfactant to enhance oil recovery in the oilfield. The screening method of this mixed bacteria has a broad scope of applications, which effectively reduces the screening cost with high accuracy, provides strong selectivity, is a convenient process and easy to operate, and has a shorter cycle compared with the current technology.

Concrete Implementation Method

Embodiments of the invention provide a concrete implementation method, as will be described in further detail below. The following implementation method is only used to explain various embodiments of the invention, rather than limiting the scope of this invention.

According to at least one embodiment, a screening method of mixed bacteria producing biosurfactant includes the following steps:

(1) Bacterial Activation: activating a bacterial strain, which includes transferring the bacterial strain for oil production on a slant culture medium, culture 2 d at 37° C., among which the slant culture medium includes beef extract 3 g, peptone 10 g, NaCl 5 g, agar 20 g, and distilled water 1000 mL, and has a pH 7.0, and a steam sterilization (121° C., 20 min).According to at least one embodiment, the bacterial strain for oil production awaiting screening in this implementation method includes, for example, Pseudomonas stutzeri with preservation No. of CCTCC AB 205091, Nocardioides ginsengagri with preservation No. of CCTCC 52013441, and Bacillus licheniformis with preservation No. of CCTCC AB 205141. As will be used in this disclosure, “CCTCC” represents the preservation center, China Center for Type Culture Collection (CCTCC). According to at least one embodiment, the bacterial strain further includes Trichoderma reesei, Pseudomonas aeruginosa, Lactobacillus rhamnosus, Methylobacterium extorquens, Thermus thermophilus, Nocardioides luteus Prauser with the preservation No. of ATCC43052, Kibdelosporangium aridum subsp. aridum Shearer with the preservation No. of ATCC39323, Desulfobacter postgatei with the preservation No. of ATCC33911, and Streptobacillus moniliformis with the preservation No. of NCTC 10651.

(2) Seed Culture: after culturing and activating on the slant culture medium, transferring the bacterial strain awaiting screening to a triangular flask of a seed liquid medium, and shakingthe cultivation (37° C., 16 h), at a rotation speed of 160 revolutions/min. According to at least one embodiment, the seed liquid medium includes: grape 5 g, beef extract 3 g, peptone 10 g, MgSO₄.7H₂O 2 g, and deionized water 1000 mL, and has a pH7.2, and a steam sterilization (121 ° C., 20min).

(3) Fermentation Culture: inoculating a 4% inoculation amount of the seed liquid medium of the seed culture in a primary fermentation medium (200 mL/500 mL) in step (2), shaking the cultivation (37° C., 72 h), at a rotation speed of 160 revolutions/min. According to at least one embodiment, the seed liquid medium includes glucose 20 g, peptone 4 g, KH₂PO₄ 5 g, K₂HPO₄ 5 g, MgSO₄.7H₂O 0.25 g, NaCl 2 g, CaCl₂ 0.08 g, and deionized water 1000mL, and has a pH7.0, and a steam sterilization (121° C., 20 min).

(4) Initial screening with blood plate method: Dibbling the bacteria strain awaiting screening after fermentation and culturing in step (3) with a sterile toothpick on a cooling blood plate separation culture medium, wherein each bacterial strain is repeated three times as a parallel experiment, cultured for 24h-48h at 37° C., preliminarily judging the bacterial strain's ability to produce a biosurfactant according to the hemolysis ring diameter on the blood plate, which is screened by using the biosurfactant's hemolytic characteristics. According to at least one embodiment, the blood plate separation culture medium includes beef extract 3 g, peptone 10 g, NaCl 5 g, agar 15 g-20 g, and distilled water 1000 mL, and as a pH 7.0-7.2, and a steam sterilization (121° C., 20 min). When the culture medium is cooled to about 45° C., 100 mL supernatant of fresh pig blood is added and homogeneously mixed.

(5) Re-screen with oil drain method: fetch dibbling the bacteria strain awaiting screening in step (4) in a blood plate separation culture vessel with a diameter, for example, of 15 cm, adding 1 mL of a liquid paraffin after adding 100 mL water, spreading a liquid paraffin into a circular oil film, adding 10 μL of a fermentation liquid that has centrifugal, removed impurities after extraction in the center, and finally measuring the diameter of an oil drain ring and tracking measurement for 5 days.

According to at least one embodiment, the bacterial strain producing surfactant screened by the above methods is comprised of three bacterial strain, which include: (1) Pseudomonas stutzeri with preservation No. of CCTCC AB 205091,collection date Nov. 21, 2015; (2) Nocardioides ginsengagri with preservation No. of CCTCC 52013441, collection date Mar. 1, 2013; and (3) Bacillus licheniformis with preservation No. of Bacillus licheniformis, collection date Oct. 21, 2005.

According to at least one embodiment the system classification and identification of mixed bacteria by the screening methods discussed above is listed as follows:

(1) Morphological identification: including single colony morphology observation, using a streak plate method of a screened bacterial strain, culture 24-48 h at 37° C., then conducting a Colony morphology observation, a Cell morphology observation, and conducting a morphological observation under an optical microscope after gram staining, spore staining, and capsule staining for bacteria.

(2) Physiological and biochemical properties identification: Starch hydrolysis experiment on the bacterial strain (Starch culture medium including beef extract 3 g, peptone10 g, NaCl 5 g, soluble starch 2 g, agar 20 g, dissolved in 1000 mL water, with a steam sterilization for 20min at 121° C.). Gelatin liquefaction experiment (Gelatin medium including gelatin medium 3 g, peptone 10 g, NaCl 5 g, dissolved in 100 mL water, add gelatin 12-18 g, melt the above components in water bath, stir constantly, modulating pH 7.2-7.4 after melting, steam sterilization for 30min at 121° C.). Litmus milk test (Litmus milk medium including milk powder 100 g, litmus 0.075 g, dissolved in 1000 mL water, with a pH6.8, and a steam sterilization for 15 min at 121° C.). Sugar fermentation experiment (Sugar fermentation medium including peptone 10 g, NaCl 5 g, dissolved in distilled water 1000 mL, modulating pH to 7.6, install the culture medium listed above respectively in the test tube with Dehanshi tubule inside), steam sterilization at 121° C. for 20 min, configure 10 mL 20% glucose and Lactose solution, then steam sterilization at 121° C. for 30 min. After sterilization for each one, add 0.5 mL 20% sterile sugar solution by aseptic operation in each tube), Methyl red test, Voges-Proskauer test, Citrate experiment (Citrate culture medium including NH₄H₂PO₄ 1 g, K₂HPO₄ 1 g, NaCl 5 g, MgSO₄ 0.2 g, sodium citrate 2 g, agar 20 g, dissolved in distilled water 1000 mL. After heating and melting the above components, modulating pH to 6.8, adding indicator 1% blue ethanol solution of bromine 10mL, filter absorbent cotton after shaking well, putting them in the test tube respectively, make slant after steam sterilization at 121° C. for 20min). Hydrogen sulfide test, Contact enzyme test, Lecithin enzyme experiment (Egg yolk agar plate medium including yeast extract 5 g, peptone 10 g, NaCl 10 g, agar 20 g, 5% yolk suspension, dissolved in 1000 mL water, modulating pH to 7.0 with NaOH of 1M, steam sterilization at 121° C. for 20 min), and Nitrate experiment (Nitrate medium including potassium nitrate 0.2 g, peptone 5 g, distilled water 1000 mL, modulating pH to 7.4, packed in a test tube, steam sterilization at 121° C. for 15 min).

(3) rDNA Sequence Analysis:

a) Extraction of genomic DNA: choose the 1500 μL bacterial suspension which cultured to several growing periods, 12000 revolutions/min, 1 min, collecting bacteria; re-suspension the bacteria in 300 μLTE buffer solution; add lysozyme 6 μL, heat preservation in 37° C. for 30 min; add 68° C. preheating 10% SDS 16.5 μL; add Protease K (20 mg/mL)18 μL, heat preservation in 55° C. for 2 h; add phenol /trichloromethane/isoamyl alcohol of equal volume (25:24:1, homogeneously mixing, 12000 revolutions/min, centrifugal 5 min, take supernatant to new centrifugal tube, repeat 2-3 times; add supernatant to 1/10 volume sodium acetate trihydrate (3 mol/L) and equal volume of isoamyl alcohol, keep −20° C. for 1.5-2 h, then 12000 revolutions/min, 5 min; add precooling 70° C. ethanol of equal volume to supernatant, 12000 revolutions/min, centrifugal 15 min, discard supernatant, put centrifugal tube in draught cupboard for drying; dissolve precipitation with 50 μL ddH2O, preserve in −20° C. for reserve.

b) PCR Amplification, reactant purification,connection and transformation: Design universal primer : Forward primer 5′-AGAGTTTGATCCTGGCTCAG-3′ (SEQ ID:1), Reverse primer 5′-GGTTACCTT GTTACGACTT-3′ (SEQ ID:2), do agarose gel electrophoresis experiment on PCR amplification products, choose MAX rubber recycling kit to purify PCR product, connect purification product with pMD18-T carrier, then transform it to x strain sensitive cell to conduct blue-white screen experiment, choose some white spots to activate, verify the transformation result by colony PCR experiment.

c) 16S rDNA Sequence Analysis: Bring the chosen positive clone to a sequencing company to conduct sequence testing, and then submit the sequence result to GenBank to do a Blast analysis and draw a phylogenetic tree, conduct a PCR amplification by a primer 16S rDNA to get a 16S rDNA with a sequence length 1500 bp, submit the sequence to NCBI for a sequence alignment, submit the gene sequence got through test to NCBI for sequence alignment, make a sequence homology comparison between a gene sequence, which has been tested and a GenBank database through a blast to obtain a gene sequence of similar typical strains, input the sequence to conduct a blast comparison search and analysis, a bidirectional measurement and sequence splicing. According to at least one embodiment, the similarity reaches 98% between 99 kinds of bacteria and the sequence one, so it can be determined to be Pseudomonas aeruginosa, in which the final identification is Pseudomonas aeruginosa.

4. Qualitative analysis methods and results of biosurfactant produced by mixed bacteria according to various embodiments of the invention:

a) Fetch thin layer chromatography silica gel and 0.4% CMC-Na solution with the proportion of 1:3 into mortar to grind homogeneously, and then put them on the glass plate to dry naturally by air, activate at 105° C. for 30 min to reserve.

b)Fermentation liquid 10000 r/min, Centrifugal 20 min, use equal volume of Trichloromethane/ Methanol (2/1, v/v) to mix and extract for 12 h as supernatant, extract for twice, take the lower layer as spotting sample.

c) Absorb the sample with capillary after centrifugal extraction for spotting.

d) Choose the mixture of trichloromethane/methanol/water (65/25/4, v/v/v) as spreading agent, fetch spreading agent with reasonable amounts to a chromatography groove, put the chromatography plate, which is spotting well in the groove (spreading agent could not be higher than the standard line), cover it well with chromatography groove lid, remove the chromatography plate, spray the chromogenic reagent after natural air drying. Three kinds of chromogenic reagent, according to various embodiments of the invention, are listed as following:
1. Phenol—sulfuric acid reagent, detecting the glycolipid surfactant, showing brown; 2. ammonium molybdate-perchloric acid chromogenic reagent, detecting type of phospholipids surfactant, showing aquamarine; and 3. 0.5% Ninhydrin chromogenic reagent, detecting lipopeptid surfactant, showing red. According to at least one embodiment, the bacterial strain is identified as Pseudomonas aeruginosa by morphology observation, Gram stain, physiological and biochemical responses, and 16s rDNA sequence Analysis.

5. Quantitative analysis method and result of biosurfactant produced by the mixed bacteria according to various embodiments of the invention:

a) Extraction of biosurfactant: fermentation liquid 10000 r/min centrifugal 20 min, take supernatant; make supernatant pH2.0 with concentrated hydrochloric acid, 4° C. sat quietly for a night; 10000 revolutions/min centrifugal 20 min, collect precipitation. After washing and precipitation with a small amount hydrochloric acid solution of pH 2.0, use 1 mol/L NaOH solution to modulate the PH of precipitation to 7.0, and freeze the dry surfactant crude sample; dissolve the crude sample in trichloromethane/methanol (2/1, v/v) solution, rotating evaporation to remove organic solution, freeze and dry it to get the biosurfactant sample.

b) Preparation of standard surfactant solution: Weigh 75 mg surfactant used in the laboratory and dissolve it into the sterile distilled water, pour it to volumetric flask, sterile distilled water with constant volume to 50 mL, and get the standard biosurfactant solution with the concentration of 1500 mg/L. Dilute it to certain times to get the standard biosurfactant solution with the concentration of 300 mg/L, 600 mg/L, 900 mg/L and 1200 mg/L, put them in the icebox at 4° C. for preservation.

c) Drawing of relationship curve between diameter of oil drain ring and concentration of biosurfactant: According the report of relevant literature, there was a linear relationship between diameter of oil drain ring and biosurfactant amount, and draw the relationship curve between diameter of oil drain ring and concentration of biosurfactant on the basis of standard concentration of biosurfactant as abscissa and diameter of oil drain ring as ordinate.

d) The precision test: Continuously measure the 1200 mg/L standard biosurfactant solution oil drain ring for 6 times, then calculate RSD value.

e) Repetitive experiment: take 6 portions of fermentation liquid of the same batch, parallel measure the size of its oil drain ring, calculate RSD value.

Activated bacterial strain is inoculated into a seed culture medium, 160 revolutions/min shake cultivation, fetch seed culture fluid every 2 h. After, dilute it and determine its absorbance in the place where wavelength is 620 nm. Take culture time as abscissa, the result is shown on FIG. 1.

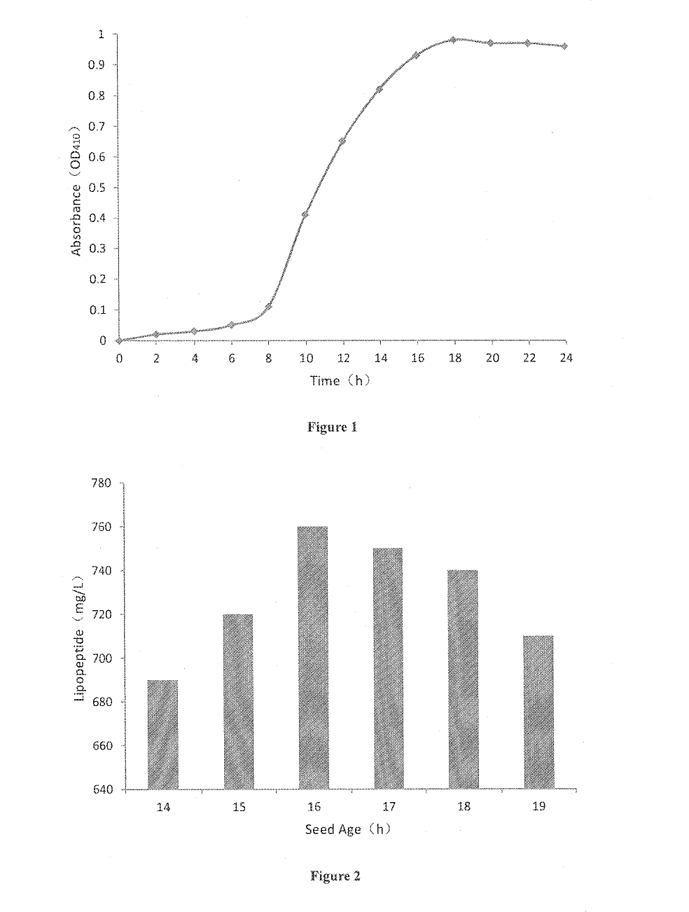

Inoculate seed liquid with seed age of 14, 15 h, 16 h, 17 h, 18 h, 19 h to primary fermentation medium with inoculation quantity of 4%, 37° C., 160 revolutions/min fermentation for 72 h, and determine the biosurfactant production amount. The result is showed on FIG. 2, from which the bacterial strain in its late stage of logarithmic growth has strong adaptability to new environment, and it could grow quickly after inoculation, which is helpful to increase the biosurfactant production. And the bacterial concentration is relatively higher at this time, which is conducive to maintain a higher inoculation quantity.

Thin-layer chromatography result: No color is shown when taking phospholipid and lipid chromogenic reagent. So the preliminary judgment is that it's not lipopeptide biosurfactant. And ninhydrin chromogenic reagent shows no color. Therefore, it can be judged that it's not lipopeptide biosurfactant. Only glycolipid chromogenic reagent has obvious brown spot. So extracted products only consists of glycolipid rather than lipid, phospholipid and lipopeptide. Please refer to table 1 for the detailed result.

TABLE 1

| Expansion agent | Proportion | phenomenon | Rf |
|---|---|---|---|
| V(trichloromethane):V(methanol): | 65:15:2 | brown spot | 0.45 |
| V(water) | | | |
| V(trichloromethane):V(methanol): | 80:25:1 | brown spot | 0.55 |
| V(acetic acid) | | | |
| V(trichloromethane):V(ethanol) | 8:2 | brown spot | 0.50 |
| V(hexane):V(ethanol):V(acetic | 80:20:1 | brown spot | 0.52 |
| acid) | | | |
| V(butyl alcohol):V(acetic acid): | 16:4:8 | brown spot | 0.48 |
| V(water) | | | |

Conduct thin layer chromatography analysis on sample extract of glycolipid chromogenic reagent and develop the spotting silica gel plate with different developing solvent, and determine Rf value on the color reaction. According to the glycolipid TLC analysis, Rf of the measured material in the test is similar to Rf of rhamnolipid. Therefore, it can be deduced that the extract is rhamnolipid.

6.The application of biosurfactant produced by mixed bacteria in oil recovery

Test the biosurfactant produced by the mixed bacteria; There are two kinds of experimental oil, interfacial tension measurement for kerosene oil, determination of viscosity ratio and preparation of oil sands for the crude oil from BQ33 Wells in the ancient city, the viscosity of the crude oil under the condition of 40° C. is 27,000 m Pa/s. The oil sands used: Experimental sands is 0.8- 1.2 mm quartz sand (white), wash and dry, put 20 g quartz sand and 3 g oil into 100 ml beaker, heat and mix well, keep the mixture in the 50° C. water for 4 days. The experiment water is distilled water.

Static Oil Displacement Test

Add 50 ml of different concentration of BERO™ solution in a 100 ml beaker of oil sands, let stand for 24 h at 50° C. temperatures, and then shake the beaker, oil escape from the oil sands, measuring the amount of oil washed out from the oil sands by the BERO™ solution. Oil displacement efficiency calculation method:

Oil displacement efficiency=(V₂/V₁)×100%

V₁—the oil content in the oil sands (ml)

V₂—oil displacement (ml)

One can see from Table 2, oil displacement efficiency increases with the increase of

BERO™ concentration, from the test results, BERO™ can work in very low concentrations (0.5%), the oil displacement efficiency is the best when the concentration of surfactant was 2%. BERO™'s oil displacement efficiency decreases after high temperature heat treatment, the specific performance is that the oil droplets are significantly larger, the compared levitation force decreased, may be the surfactant activity decreased after heat treatment.

TABLE 2
Determination data tables of the oil wash effect after treated by different
concentrations of surfactant solution:

| | Concentration (%) | | | | | | | |
|---|---|---|---|---|---|---|---|---|
| | 0 | 0.5 | 1 | 1.5 | 2 | 2.5 | 3 | 2* |
| Oil | 38.7 | 83.2 | 85.5 | 91.4 | 94.4 | 93.3 | 94.2 | 83.4 |
| displacement | | | | | | | | |
| efficiency (%) | | | | | | | | |

*During the test, the 2% BERO ™ solution sealed in a stainless steel container and heat at 220° C. for 24 h.

Static Oil Wash Effect

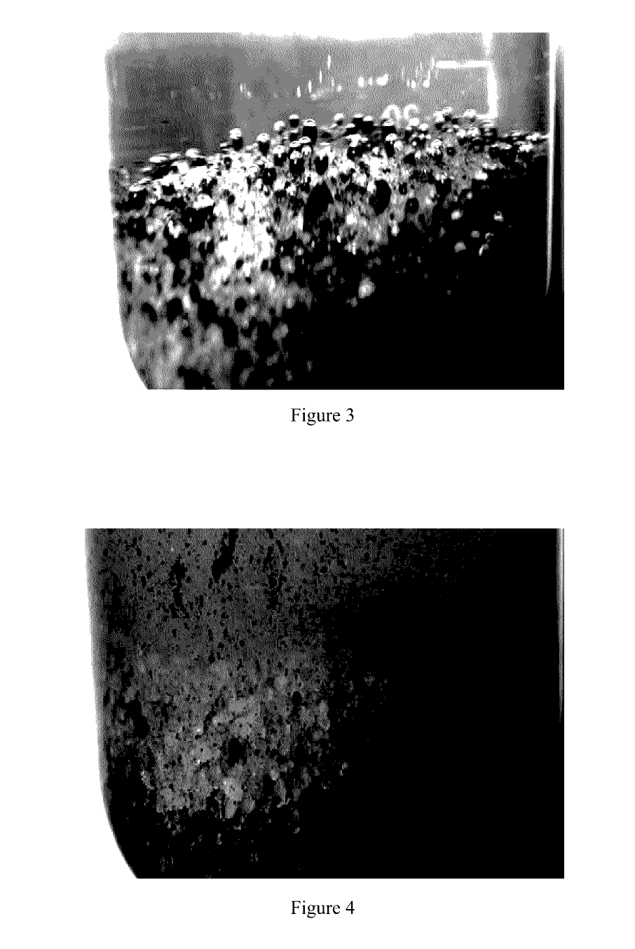

FIGS. 3 and 4 are the experiment phenomenon in the process of static wash oil. One can be seen from FIG. 3 that the crude oil in the oil sands without BERO™ curled into drops and adhere on the oil sands surface, only a small amount of floating in the water, a large number of oil drops are stuck inside the oil sands, oil displacement effect is low. Some oil drops carrying fine sand float in the water. One can see in FIG. 4, the crude oil adhered on the oil sands with BERO™ dispersed as tiny oil drop and the oil drop continuous precipitation from the oil sands, quartz sand surface is clean, oil displacement effective is high. The reason could be that the BERO™ molecules can be attached to the surface of the oil sands and split the oil drops from the sand surface. Part of the BERO™ carrying oil mixed with water, the other part of the BERO™ molecules are attached to the surface of sand and make the other oil molecules can't attach to this part of the sand. The enhanced oil characteristics of BERO™ mixed with the water began to work, it quickly separate the oil from the sand, because the BERO™ itself insoluble in oil, but soluble in water, it stay in the water and continue the process.

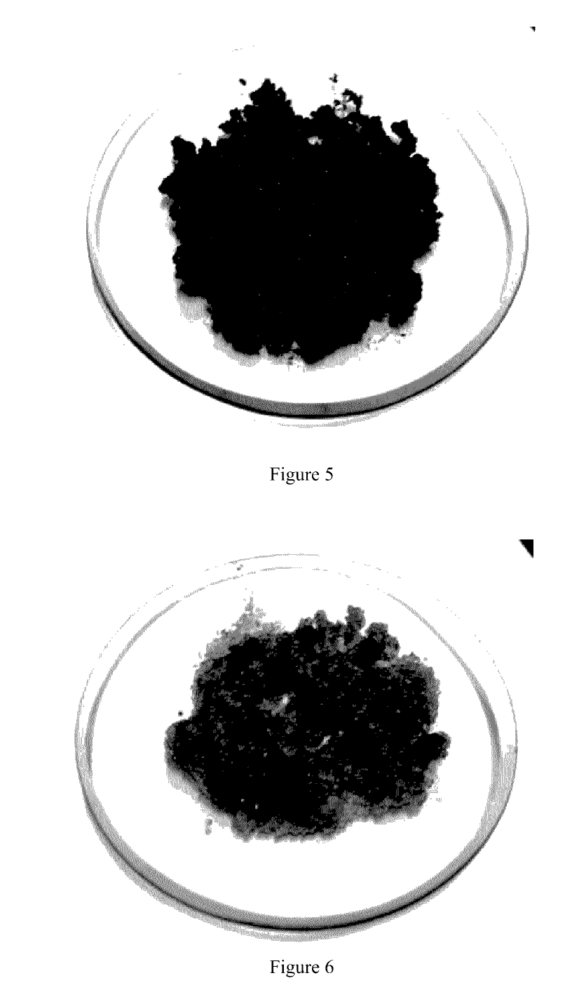

FIGS. 5 and 6 are pictures of the residual oil sands after the completion of the experiment. As shown in FIG. 5, there is a large amount of the residual oil staying in the oil sand after water wash, quartz sands form conglobation. As shown in FIG. 6, after washing by BERO™, the oil sands seem loose and clean. Therefore, the BERO™ oil washing effect is remarkable. It changed the oil sands surface to be wettability and prevent crude oil from adhering to the sands.

Measurement of the Surface Tension and Interfacial Tension

The following table shows the surface tension and interfacial tension of different concentrations of surfactant solution, the test result is the average value measured at room temperature with K12 type surface tension meter.

TABLE 4
Measurement date of the surface tension of different
concentrated BERO ™ solution:

| Viscosity of | Surface | Interfacial |
|---|---|---|
| Sample (%) | tension (mN/m) | tension (mN/m) |
| 0 | 73.94 | 36.52 |
| 0.5 | 44.44 | 1.88 |
| 1 | 44.49 | 2.96 |
| 1.5 | 46.00 | 2.71 |
| 2 | 44.56 | 3.10 |
| 3 | 45.01 | 2.72 |

Put the BERO™ solution in sealed stainless steel containers, with heat treatment at 220° C. for 24 hours, then carried out and measured the surface tension and interfacial tension at different concentrations of BERO™ solution, Table 5 shows the measurement data.

TABLE 5
measurement data of the surface tension and interfacial tension at
different concentrations of surfactant solution after heat treatment:

| Viscosity of | Surface | Interfacial |
|---|---|---|
| Sample (%) | tension (mN/m) | tension (mN/m) |
| 0 | 73.94 | 36.52 |
| 0.5 | 40.12 | 2.95 |
| 1 | 42.10 | 3.32 |
| 1.5 | 42.49 | 3.51 |
| 2 | 42.49 | 3.98 |
| 3 | 45.24 | 4.29 |

From Table 4, one can see the BERO™ solution can greatly reduce the surface tension and interfacial tension of oil-water, and when the concentration was reduced, surface tension and interfacial tension are decreasing, it explains that the enzymes have good effect even in low concentrations, can change the wettability of rock surface, reduce the flow resistance of oil.

As shown in Table 5, the surface tension and interfacial tension of the BERO™ before or after the 24 hours of heat treatment at 220° C. were similar, it indicates that the BERO™ has strong heat resistance performance and can be used for thermal recovery of heavy oil.

BERO™ Corrosion Test

BERO™ liquid PH value of 5.5 2%, the concentration of BERO™ aqueous solution PH value of 6.5, is a weak acid. In order to investigate the corrosion of surfactant on the steel body, adopted N80 tube with grinding and clear surface as test material, put the specimens in the 2% concentrated BERO™ solution, sealed and keep still under 90° C. oven for 24 h, observe the phenomenon of the specimen surface. Experimental results show that most of the specimen surface brightness, but part of the specimen surface had black deposit, after removing the sediment found a shallow corrosion pit, so the BERO™ solution is slightly corrosive.

The above tests illustrate that the BERO™ can work under the condition of low concentrations (0.5%), the oil displacement efficiency is the best when the BERO™ concentration is 2%. The BERO™ can release oil from the surface of oil sands, the oil sands become loose and clean after washing, and it can prevent the re-adhesion of the crude oil and sands. The BERO™ has no viscosity reduction function. The main effect is that BERO™ biosurfactant can peel off gathered heavy oil to form a dilute and soft oil flow zone. The BERO™ solution can greatly reduce the surface tension and interfacial tension of the oil-water, and has good resistance to high temperature. The BERO™ solution is in weak acid, at high temperature it will slightly corrode the N80 specimens.

The biosurfactant produced by mixed bacteria according to various embodiments of the invention is a biological surface active catalyst preparation extracted from microbial biochemistry, and it has direct, quick response compared with microbial biochemistry in enhanced oil recovery, according to conventional methods, which is the most advanced technology in enhancing oil recovery in the oilfield in China and abroad. The biosurfactant produced by mixed bacteria according to various embodiments of the invention has the advantages such as wide application area, simple process, less investment, quick effect, no damage to the oil layer and no pollution, etc., while enabling low permeability old oil field to gain enhanced production.

Biosurfactant is the leading technology in the microorganism oil production technology, which uses the modern bioengineering technology such as gene engineering, cell engineering, enzyme engineering, etc., which could efficiently release hydrocarbon (oil) on the surfaces of solid particles. Meanwhile, it's an efficient, environmentally friendly liquid products to release crude oil on the surfaces of solid particles, which could conduct biological degradation to efficiently release hydrocarbon (crude oil) on the surfaces of solid particles. After being injected into a stratum, biosurfactant can rapidly strip wax and asphalt crystallized and accumulated on rock particles in the area of a reservoir close to well and part of BERO™ adheres to rock surface to make the wettability of the rock become water-wetting, lower the flowing resistance of crude oil in formation voids, thus releasing crude oil from rock particle surface and being separated out from micropores. Biosurfactant molecules entering into water can be transported to the area further than the surrounding area of sandstone formation and form new oil outlet passages in sandstone formation so as to clean production well in oil reservoir, plugging removal and injection reduction of water injection well, plugging removal and stimulation, displacement and improving oil recovery rate.

Embodiments of the invention may suitably comprise, consist or consist essentially of the elements disclosed and may be practiced in the absence of an element not disclosed. For example, it can be recognized by those skilled in the art that certain steps can be combined into a single step.

Unless defined otherwise, all technical and scientific terms used have the same meaning as commonly understood by one of ordinary skill in the art to which this invention belongs.

The singular forms “a,” “an,” and “the” include plural referents, unless the context clearly dictates otherwise.

As used herein and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur. As used herein, terms such as “first” and “second” are arbitrarily assigned and are merely intended to differentiate between two or more components of an apparatus. It is to be understood that the words “first” and “second” serve no other purpose and are not part of the name or description of the component, nor do they necessarily define a relative location or position of the component. Furthermore, it is to be understood that the mere use of the term “first” and “second” does not require that there be any “third” component, although that possibility is contemplated under the scope of the embodiments of the present invention.

Ranges may be expressed herein as from about one particular value, and/or to about another particular value. When such a range is expressed, it is to be understood that another embodiment is from the one particular value and/or to the other particular value, along with all combinations within said range.

All publications mentioned are incorporated by reference to disclose and describe the methods or materials, or both, in connection with which the publications are cited. The publications discussed are provided solely for their disclosure prior to the filing date of the present application. Nothing is to be construed as an admission that the invention is not entitled to antedate such publication by virtue of prior invention. Further, the dates of publication provided may be different from the actual publication dates, which may need to be independently confirmed.

Although the present invention has been described in detail, it should be understood that various changes, substitutions, and alterations can be made hereupon without departing from the principle and scope of the invention. Accordingly, the scope of the present invention should be determined by the following claims and their appropriate legal equivalents.

1. A mixed bacteria producing biosurfactant, comprising:
three strains comprising:
(1) Pseudomonas stutzeri, preservation No.: CCTCC AB 205091;
(2) Nocardioides ginsengagri, preservation No.: CCTCC S2013441; and
(3) Bacillus licheniformis, preservation No.: CCTCC AB 205141.

2. A screening method of a mixed bacteria producing biosurfactant, the method comprising the steps of:
selecting a bacterial strain for oil production awaiting screening;
activating and culturing the selected bacterial strain for oil production awaiting screening;
selecting the bacterial strain for oil production awaiting screening which is activated and cultured to obtain a primary screening fermentation liquid using a blood plate method; and
re-screening the primary screening fermentation liquid to obtain the mixed bacteria producing biosurfactant using an oil drain method.

3. The screening method of the mixed bacteria producing biosurfactant according to claim 2, wherein the selected bacterial strain is comprised of three strains including Pseudomonas stutzeri with preservation No. of CCTCC AB 205091, Nocardioides ginsengagri with preservation No. of CCTCC S2013441, and Bacillus licheniformis with preservation No. of CCTCC AB 205141.

4. The screening method of the mixed bacteria producing biosurfactant according to claim 2, wherein the activating step comprises transferring the bacterial strain for oil production awaiting screening onto a slant medium, and culturing the slant medium for 2 days at 37° C., wherein the slant medium is comprised of beef extract 3 g, peptone 10 g, NaCl 5 g, agar 20 g, and distilled water 1000 mL, and has a pH of 7.0, and a steam sterilization of 121° C., 20 minutes.

5. The screening method of the mixed bacteria producing biosurfactant according to claim 2, wherein the culturing step comprises seed culturing and fermentation culturing in order.

6. The screening method of the mixed bacteria producing biosurfactant according to claim 5, wherein the seed culturing step comprises transferring the bacterial strain's seed awaiting screening after the activation and culturing the selected bacterial strain, to a seed liquid medium, shaking the cultivation at 37° C. for 16 hours using a rotational speed of 160 revolutions/minute, wherein the seed liquid medium is comprised of grape 5 g, beef extract 3 g, peptone 10 g, MgSO₄.7H₂O 2 g, and deionized water 1000 mL, and has a pH of 7.2, and a steam sterilization of 121° C., 20minutes.

7. The screening method of the mixed bacteria producing biosurfactant according to claim 5, wherein the fermentation culturing step comprises inoculating a 4% inoculation amount of a seed liquid produced by seed culturing in a primary fermentation medium, shaking the cultivation at 37° C. for 72 hours using a rotational speed of 160revolutions/minute, wherein the primary fermentation medium is comprised of glucose 20 g, peptone 4 g, KH₂PO₄ 5 g, K₂HPO₄ 5 g, MgSO₄7H₂O 0.25 g, NaCl 2 g, CaCl₂ 0.08 g, and deionized water 1000 mL, and has a pH of 7.0, and a steam sterilization of 121° C., 20 minutes.

8. The screening method of the mixed bacteria producing biosurfactant according to claim 2, wherein the blood plate method comprises dibbling the bacterial strain awaiting screening with a sterile toothpick on a cooling blood plate separation culture medium, culturing the bacterial strain for 24 h-48 h at 37° C. to determine the bacterial strain's ability to produce biosurfactant according to a hemolysis ring diameter on the blood plate, wherein the blood plate separation culture medium is comprised of beef extract 3 g, peptone 10 g, NaCl 5 g, agar 15 g-20 g, distilledwater 1000 mL, and has a pH of 7.0-7.2, and a steam sterilization of 121° C., 20 minutes; the blood plate method further comprising, adding 100 mL of supernatant of fresh pig blood and homogeneously mixing, when the blood plate separation culture medium is cooled to about 45° C.

9. The screening method of the mixed bacteria producing biosurfactant according to claim 2, wherein the oil drain method comprises fetch dibbling the bacterial strain awaiting screening and a blood plate separation culture vessel with diameter 15 cm, adding 1 mL of a liquid paraffin after adding 100 mL water, when the liquid paraffin spreads into a circular oil film, adding 10 μL of a fermentation liquid that has centrifugal, removed impurities, after extraction in the center, and measuring the diameter of an oil drain ring and tracking measurements for 5 days.

10. A biosurfactant extracted and processed by mixed bacteria according to claim 1.

11. The biosurfactant extracted and processed by mixed bacteria according to claim 10, wherein the main ingredient of the biosurfactant is rhamnolipid.

12. The biosurfactant extracted and processed by mixed bacteria according to claim 10, wherein the physical and chemical index of the biosurfactant is a concentrated liquid having a brown translucent appearance and enzyme odor, a PH of 5-7, a density of 1-1.1 g/cm³, is completely soluble in water and compatible with any degree of mineralization of sewage, is insoluble in oil, and has a boiling point 100° C. and temperature resistance <220° C.

13. The application of the biosurfactant extracted and processed by mixed bacteria according to claim 10 in an oilfield for recovery of oil.

14. The application of the biosurfactant extracted and processed by mixed bacteria according to claim 13, wherein the application density of the biosurfactant is 0.5-3.0 wt. %.

15. The application of the biosurfactant extracted and processed by mixed bacteria according to claim 14, wherein the application density of the biosurfactant is 2.0 wt %.

16. The application of the biosurfactant extracted and processed by mixed bacteria according to claim 13, wherein the biosurfactant is applied to a working environment no higher than 220° C.

17. The application of the biosurfactant extracted and processed by mixed bacteria according to claim 13, wherein the biosurfactant is applied to enhance production for a heave oil thermal recovery well.
